(19) United States
(12) Patent Application Publication
(10) Pub. No.: US 2014/0107549 A1
(43) Pub. Date: Apr. 17, 2014

(54) INFLATABLE WRAP WITH AUTOMATIC TIGHTNESS DETECTION

(71) Applicant: CAREMED SUPPLY INC., New Taipei City (TW)

(72) Inventor: Pao Ming Shih, Taipei City (TW)

(73) Assignee: CAREMED SUPPLY INC., New Taipei City (TW)

(21) Appl. No.: 13/653,393

(22) Filed: Oct. 16, 2012

(51) Int. Cl. A61F 5/00

(52) U.S. Cl. 602/13

(57) ABSTRACT

An inflatable wrap with automatic tightness detection includes a wrap strap having at least one inflatable sealed zone formed thereon, at least one fluid guiding tube connected to between the sealed zone and an external fluid supply, at least two mating connection elements separately provided on two longitudinally opposite ends of the wrap strap, and a sensor module embedded in the wrap strap. The sensor module includes an output element and a sensor element arranged at locations separately corresponding to the two mating connection elements, and a processor element electrically connected to the sensor element. When the two mating connection elements are detachably connected together to bind the wrap strap around a user's body area with the sensor element and the output element becoming aligned with each other to provide an alignment sensing function, it means the wrap strap forms a closed loop structure to provide a proper pressing force.

### FIELD OF THE INVENTION

The present invention relates to an inflatable wrap for applying a pressing force to a user's selected body area, and more particularly to an inflatable wrap with automatic tightness detection to ensure proper pressing effect thereof.

### BACKGROUND OF THE INVENTION

A bedridden patient, particularly a patient after a surgical operation, tends to have slowed or even stopped blood circulation at legs, due to lying on bed over a long time. Stopped blood circulation at legs would cause thrombosis, which would further cause necrotic leg tissue or even death of patient in some worse conditions. Therefore, it is very important for patients who are suffering from edema, poor blood circulation, deep vein thrombosis or the like to move their leg muscles under help in order to improve the blood circulation thereof.

Currently, there is a type of compression wrap available in the market for applying a compressing force to a selected area on a patient's body. Such compression wrap includes a compressing wrap strap for wrapping around a patient's leg or other selected body area. The wrap strap internally includes a plurality of separated inflatable chambers, which are independently connected to a pressurized fluid supply. Generally speaking, the compressing wrap strap applies pressure first to the patient's body area, such as the patient's ankle, that is most distant from the heart and then gradually applies the pressure to other body areas closer to the heart. To use the compression wrap, first fully inflate all the chambers in the wrap strap and maintain the chambers in the fully inflated state for a period of time before deflating them at the same time, so that the wrap strap applies pulsating pressure to the patient's leg intermittently. By repeatedly applying such pulsating pressure for a period of time, the patient's blood can flow back to the heart to improve the patient's blood circulation and avoid the forming of thrombosis.

While the above-described compression wrap is useful in solving the problem of thrombosis frequently suffered by bedridden patients, most patients using such compression wrap are not certain whether the wrap straps have been properly tightened around their legs, for example. The wrap strap might not provide proper compressing pressure to effectively help moving the patient's leg muscle if it is too loosely wrapped around the patient's leg. On the other hand, the wrap strap might adversely apply too much compressing force and hurt the patient's leg if it is too tightly wrapped around the patient's leg. In brief, with the conventional inflatable wrap, the patient could not always quickly verify whether the wrap strap has been correctly wrapped around the leg or other selected body area to provide proper compressing pressure thereto. Therefore, it is desirable to develop an inflatable wrap with automatic tightness detection.

### SUMMARY OF THE INVENTION

A primary object of the present invention is to provide an inflatable wrap for applying a pressing force to a selected area on a user's body and having an automatic tightness detection ability to ensure that the inflatable wrap has been exactly wrapped around the selected body area to apply a proper pressing force thereto.

To achieve the above and other objects, the inflatable wrap with automatic tightness detection according to the present invention includes a wrap strap formed from a first sheet member and a second sheet member, which are connected to each other via at least one sealing line to thereby define at least one inflatable sealed zone between them; at least one fluid guiding tube having one end connected to and communicable with the at least one sealed zone and another end connected to an external pressurized fluid supply for supplying a fluid into the sealed zone, so that the sealed zone is expanded to apply a pressing force to a selected area on a user's body; at least two mating connection elements, including a male and a female structure, separately provided on two longitudinally opposite ends of the wrap strap for selectively detachably connecting the two opposite ends of the wrap strap to each other; and at least one sensor module embedded in the wrap strap and including an output element, a sensor element and a processor element. The output element and the sensor element embedded in the wrap strap are separately located in the two longitudinally opposite ends of the wrap strap, and the processor element is electrically connected to the sensor element. When the wrap strap is connected at the two opposite ends to form a loop structure around the user's selected body area and the sensor element and the output element are aligned with each other to provide an alignment sensing function, it is able to ensure the loop structure has a predetermined size to provide a proper pressing force on the user's body area.

In an operable embodiment of the present invention, the sensor element is in the form of a strip sensor extended parallel to a longitudinal direction of the wrap strap, so that the output element can be aligned with an optimal position on the strip sensor according to the user's body size for the wrap strap to be properly wrapped around the user's selected body area.

According to the present invention, the sensor element and the output element are embedded in the wrap strap at locations separately corresponding to the two mating connection elements; and the fluid guiding tube is extended from the at least one sealed zone into the wrap strap at a location near the sealed zone and extended out of the wrap strap at a location near a longitudinal side of the wrap strap.

In a preferred embodiment, the two mating connection elements are repeatedly usable and can be a hook tape and a loop tape of a hook-and-loop fastener, a hook and an eyelet of a hook-on fastener, or two half parts of a snap fastener. Further, the sealed zone further includes at least one bonded portion to internally divide the sealed zone into a plurality of inflatable spaces.

The present invention is characterized in that the wrap strap includes a sensor module embedded therein, which has an input element and a sensor element arranged at positions separately corresponding to the two mating connection elements provided at two longitudinally opposite ends of the wrap strap. When the wrap strap is wrapped around a selected user body area, an alignment sensing ability provided by the output element and the sensor element automatically detects whether the wrap strap has been exactly wrapped around the selected user body area with proper tightness, so as to make sure the inflatable wrap strap provides proper pressing force against the user's body area to improve the user's arterial and venous blood circulation.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIGS. 1A and 1B are front and rear views, respectively, of an inflatable wrap with automatic tightness detection according to a first preferred embodiment of the present invention;

FIGS. 2A and 2B are front and rear views, respectively, of an inflatable wrap with automatic tightness detection similar to that in FIGS. 1A and 1B but having two mating connection elements configured as two half parts of a snap fastener;

FIGS. 3A and 3B are front and rear views, respectively, of an inflatable wrap with automatic tightness detection similar to that in FIGS. 1A and 1B but having two mating connection elements configured as a hook and an eyelet of a hook-on fastener;

FIG. 4 is a front view of an inflatable wrap with automatic tightness detection according to a second preferred embodiment of the present invention; and

FIG. 5 shows the inflatable wrap of the present invention being wrapped around a user's leg.

### DETAILED DESCRIPTION OF THE PREFERRED EMBODIMENTS

The present invention will now be described with some preferred embodiments thereof and with reference to the accompanying drawings. For the purpose of easy to understand, elements that are the same in the preferred embodiments are denoted by the same reference numerals.

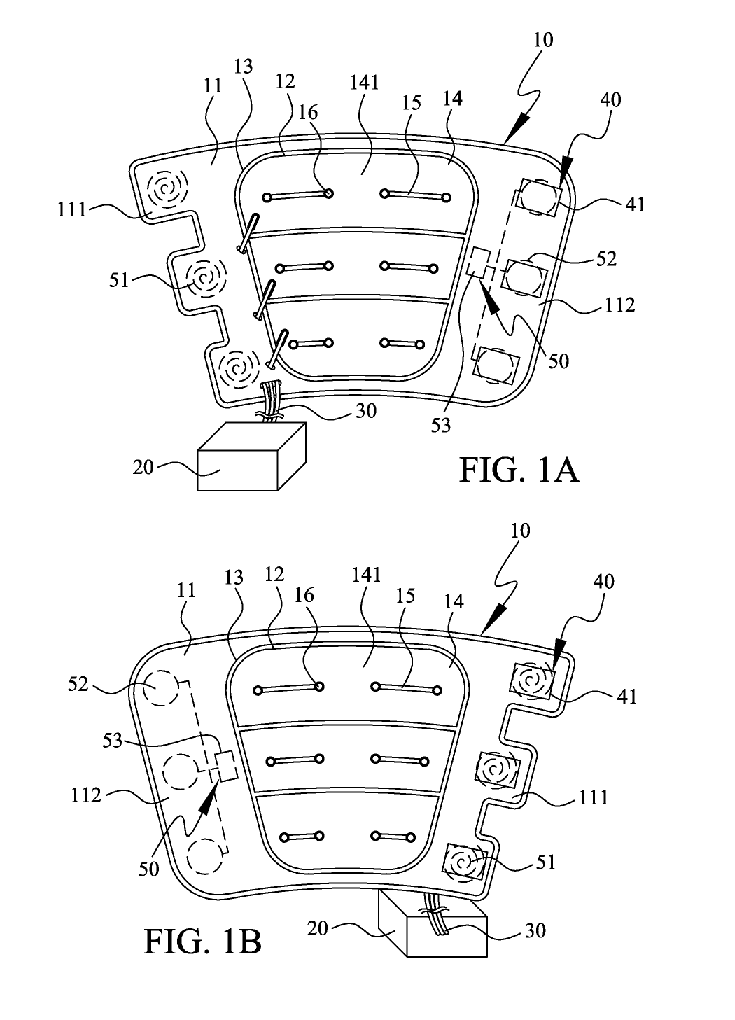

Please refer to FIGS. 1A and 1B that are front and rear views, respectively, of an inflatable wrap with automatic tightness detection according to a first preferred embodiment of the present invention. As shown, the inflatable wrap includes a wrap strap 10 for applying a pressing force to a selected area on a user's body; a fluid supply 20 for providing a pressurized fluid, such as a compressed gas; at least one fluid guiding tube 30 connected to between the fluid supply 20 and the wrap strap 10; at least two mating connection elements 40 for selectively detachably connecting two opposite ends of the wrap strap 10 to each other; and at least one sensor module 50 for detecting whether the two mating connection elements 40 have been properly connected to each other.

The wrap strap 10 is formed from a larger first sheet member 11 and a smaller second sheet member 12, which are connected to each other via at least one sealing line 13 to thereby define at least one inflatable sealed zone 14 between them. In the illustrated first preferred embodiment, the first sheet member 11 is in the form of a long strip and has three spaced attaching sections 111 forward extended from one of two longitudinally opposite ends thereof. The second sheet member 12 is located on and connected to a central area of the first sheet member 11 via three sealing lines 13, so that three adjacent and longitudinally extended sealed zones 14 are formed on the central area between the first sheet member 11 and the second sheet member 12. In each of the sealed zones 14, there is provided at least one bonded portion 15 to internally divide each sealed zone 14 into a plurality of inflatable spaces 141. Further, a through hole 16 is formed on at least one of two ends of the bonded portion 15.

The fluid guiding tube 30 has its one end connected to the sealed zone 14 and another opposite end connected to the fluid supply 20. Fluid supplied by the fluid supply 20 is guided by the fluid guiding tube 30 into the sealed zone 14 on the wrap strap 10, so that the sealed zone 14 is inflated and expanded to apply a degree of pressing force to a selected area on a user's body. In the illustrated first preferred embodiment, there are three fluid guiding tubes 30 separately extended from the sealed zones 14 into the wrap strap 10 at locations near the sealed zones 14 and extended out of the wrap strap 10 to the fluid supply 20 at a location near one longitudinal side of the wrap strap 10.

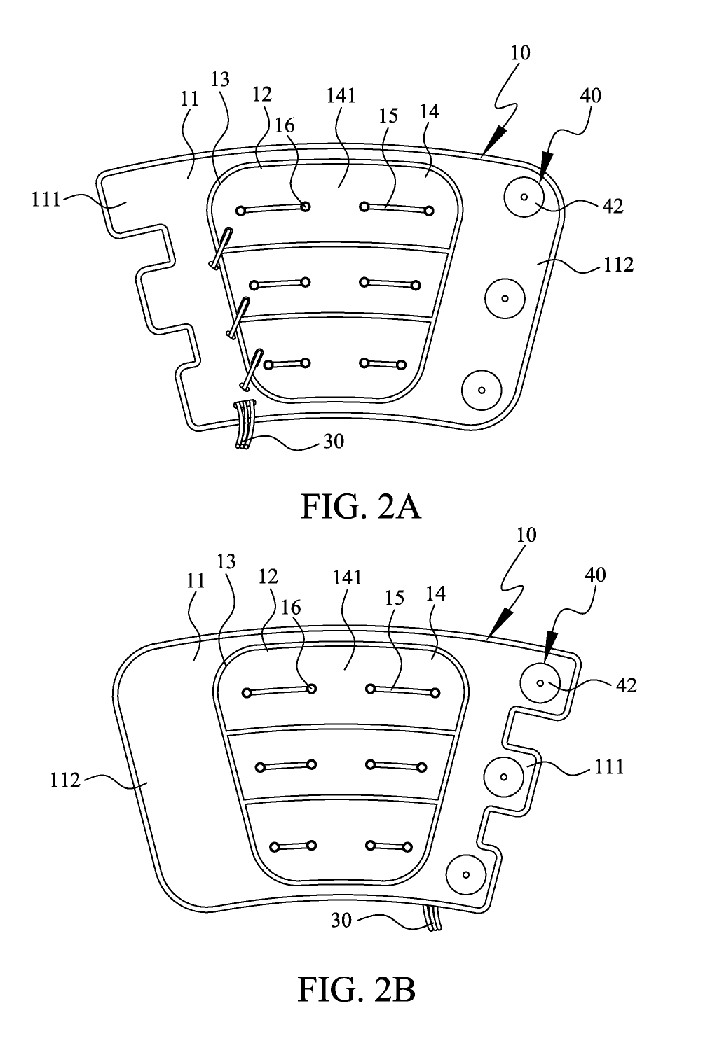

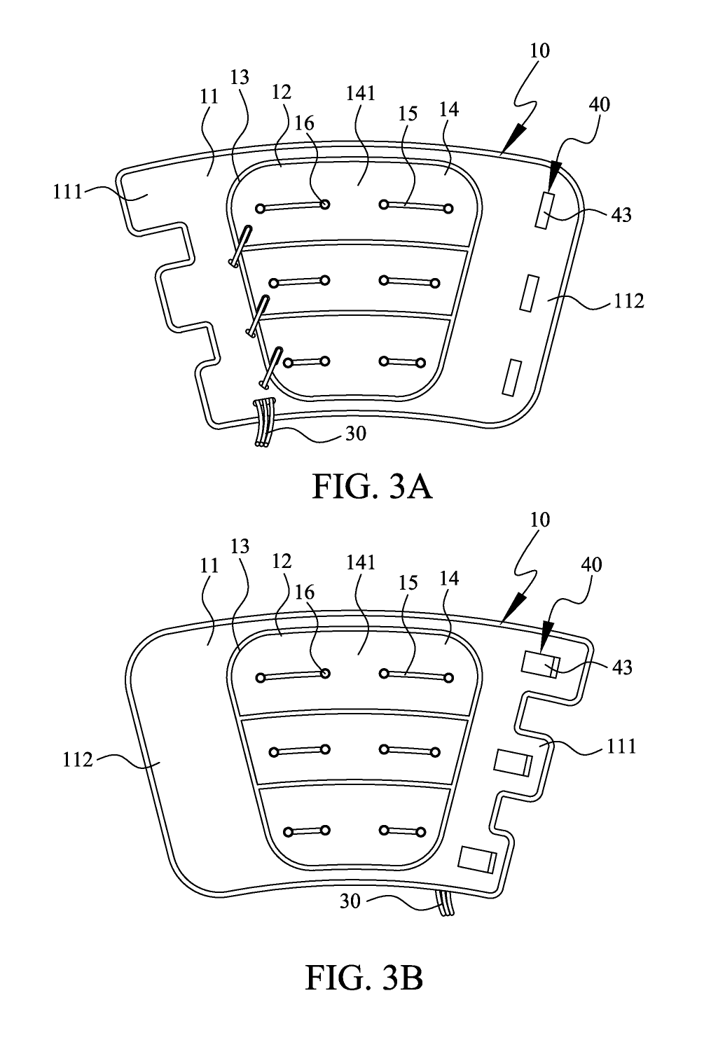

The two mating connection elements 40 include a male and a female structure, which are separately provided on the front side and the rear side of the wrap strap 10 at two longitudinally opposite ends thereof. In the illustrated first preferred embodiment, three male structures are provided on one side of the wrap strap 10 at the attaching sections 111 and three female structures are provided on the other side of the wrap strap 10 at the other longitudinal end 112 thereof. The two mating connection elements 40 shown in FIGS. 1A and 1B are two repeatedly usable mating binding elements 41, such as a hook tape and a loop tape of a hook-and-loop fastener. However, it is understood FIGS. 1A and 1B are only illustrative to enable convenient description of the present invention and not intended to limit the type of the male and the female structure for the connection element 40. For example, as shown in FIGS. 2A and 2B, the male and the female structure can be otherwise two half parts of a snap fastener 42. Alternatively, as shown in FIGS. 3A and 3B, the male and the female structure can be a hook and an eyelet of a hook-on fastener 43.

The sensor module 50 is embedded in the wrap strap 10, and includes at least one output element 51, at least one sensor element 52 and a processor element 53. The output element 51 and the sensor element 52 are separately located at the two longitudinally opposite ends of the wrap strap 10. The processor element 53 is electrically connected to the sensor element 52. In the illustrated first preferred embodiment, there are shown three output elements 51 and three sensor elements 52; and these sensor elements 52 and output elements 51 are embedded in the wrap strap 10 at the two longitudinal opposite ends thereof to separately align with the male and the female structures of the connection elements 40.

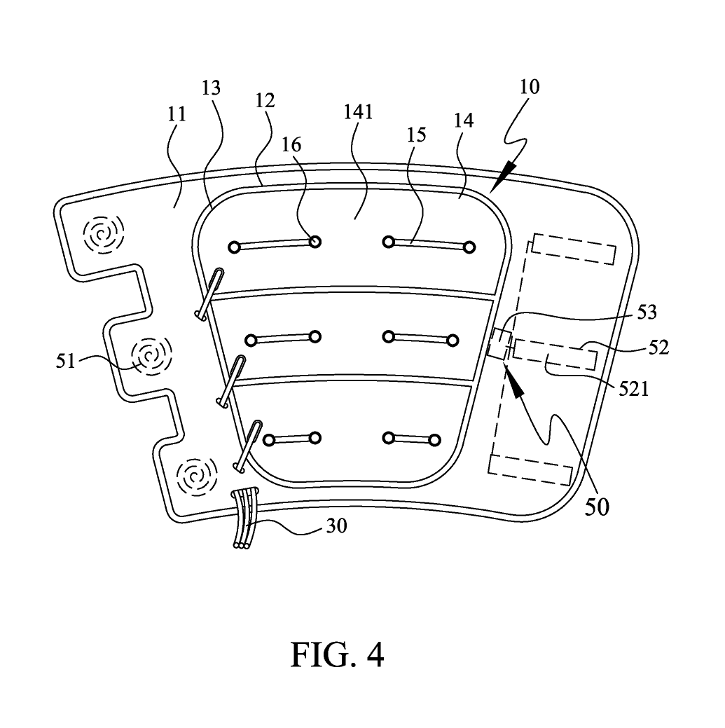

Please refer to FIG. 4 that is a front view of an inflatable wrap with automatic tightness detection according to a second preferred embodiment of the present invention. While the sensor elements 52 of the sensor module 50 in the first preferred embodiment are configured as fixed-point sensors for detecting correct connection of the male and the female structures of the connection elements to one another at the fixed points defined by the sensor elements 52, the sensor elements 52 in the second preferred embodiment are configured as parallelly spaced strip sensors 521 extended in a longitudinal direction of the wrap strap 10. With the sensor strips 521, the output elements 51 can be aligned with an optimal position on their corresponding strip sensors 521 according to different patient sizes to detect proper wrapping of the wrap strap 10 around the selected area on the patient's body.

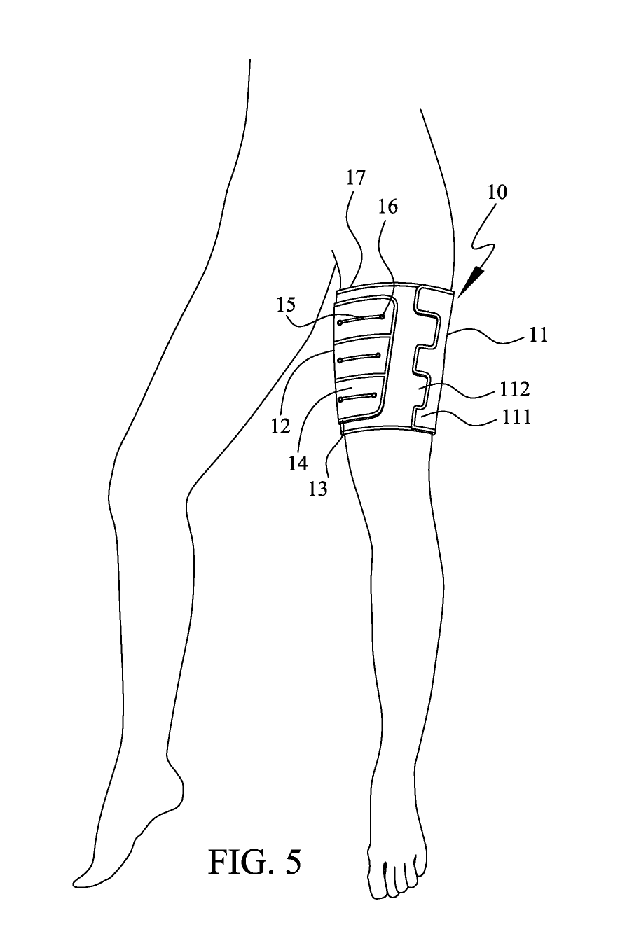

Please refer to FIG. 5. The inflatable wrap according to the present invention is characterized in that the inflatable wrap strap 10 can be quickly and correctly closed around a selected area on a user's body, such as the user's leg, to form a loop structure 17 that provides a proper pressing force against the user's body area, and that the sensor elements 52 with the output elements 51 provides an alignment sensing function, which allows the user to verify whether the inflatable wrap strap 10 is closed around the user's body area with proper tightness. Therefore, the present invention is more convenient and practical for use.

In summary, the wrap strap of the present invention includes a sensor module embedded therein, which has input elements and sensor elements arranged at positions corresponding to the mating connection elements located at two longitudinally opposite ends of the wrap strap. When the wrap strap is wrapped around a user's body area, the output elements and the sensor elements provide an alignment sensing function to automatically detect whether the wrap strap has been exactly wrapped around the selected user body area with proper tightness, so as to make sure the inflatable wrap strap provides a proper pressing force against the user's body area to improve the user's arterial and venous blood circulation.

The present invention has been described with some preferred embodiments thereof and it is understood that many changes and modifications in the described embodiments can be carried out without departing from the scope and the spirit of the invention that is intended to be limited only by the appended claims.

What is claimed is:

1. An inflatable wrap with automatic tightness detection, comprising:
a wrap strap being formed from a first sheet member and a second sheet member; the first and the second sheet member being connected to each other via at least one sealing line to thereby define at least one inflatable sealed zone between them;
at least one fluid guiding tube having an end connected to and communicable with the at least one sealed zone;
at least two mating connection elements being separately provided on two longitudinally opposite ends of the wrap strap for selectively detachably connecting the two longitudinally opposite ends of the wrap strap to each other; and
at least one sensor module including an output element, a sensor element and a processor element embedded in the wrap strap; the output element and the sensor element embedded in the wrap strap being separately located in the two longitudinally opposite ends of the wrap strap; and the processor element being electrically connected to the sensor element.

2. The inflatable wrap with automatic tightness detection as claimed in claim 1, wherein the sensor element and the output element of the sensor module are aligned with each other and provide an alignment sensing function when the two mating connection elements are properly connected together and the wrap strap is closed to form a loop structure having a predetermined size, so as to ensure the wrap strap provides a proper magnitude of pressing force.

3. The inflatable wrap with automatic tightness detection as claimed in claim 1, wherein the sensor element is configured as a strip sensor extended parallel to a longitudinal direction of the wrap strap; and the output element being aligned with an optimal position on the strip sensor according to a patient's body size to provide an alignment sensing function, so as to ensure the wrap strap is properly wrapped around a selected area on the patient's body.

4. The inflatable wrap with automatic tightness detection as claimed in claim 1, wherein the sensor element and the output element are separately located in the two longitudinally opposite ends of the wrap strap at positions corresponding to and aligned with the two mating connection elements.

5. The inflatable wrap with automatic tightness detection as claimed in claim 1, wherein the at least one fluid guiding tube is connected at another end to a pressurized fluid supply.

6. The inflatable wrap with automatic tightness detection as claimed in claim 1, wherein the at least one fluid guiding tube is extended from the at least one sealed zone into the wrap strap at a location near the sealed zone and extended out of the wrap strap at a location near a longitudinal side of the wrap strap.

7. The inflatable wrap with automatic tightness detection as claimed in claim 1, wherein the two mating connection elements are repeatedly usable and selected from the group consisting of a hook tape and a loop tape of a hook-and-loop fastener, a hook and an eyelet of a hook-on fastener, and two half parts of a snap fastener.

8. The inflatable wrap with automatic tightness detection as claimed in claim 1, wherein the sealed zone further includes at least one bonded portion to internally divide the sealed zone into a plurality of inflatable spaces.
